(19) United States
(12) Patent Application Publication
(10) Pub. No.: US 2020/0330459 A1
(43) Pub. Date: Oct. 22, 2020

(54) METHODS AND PHARMACEUTICAL COMPOSITIONS FOR THE TREATMENT OF AGE-RELATED CARDIOMETABOLIC DISEASES

(71) Applicants: INSERM (INSTITUT NATIONAL DE LA SANTÉ ET LA RECHERCHE MÉDICALE), Paris (FR); UNIVERSITÉ PARIS-EST CRÉTEIL VAL DE MARNE, Creteil f (FR); ASSISTANCE PUBLIQUE-HÔPITAUX DE PARIS (APHP), Paris (FR); OSAKA UNIVERSITY, Osaka (JP)

(72) Inventors: Geneviève DERUMEAUX, Creteil (FR); Daigo SAWAKI, Creteil (FR); Gabor CZIBIK, Creteil (FR); Takehiko YOSHIMITSU, Creteil (FR)

(21) Appl. No.: 16/089,842

(22) PCT Filed: Apr. 5, 2017
(86) PCT No.: PCT/EP2017/058159

§ 371 Date: Sep. 28, 2018

(30) Foreign Application Priority Data
Apr. 6, 2016 (EP) 16305397.8

(51) Int. Cl. A61K 31/4985; C12N 15/113; A61K 39/395; A61P 9/00; A61P 3/00

(52) U.S. Cl. CPC A61K 31/4985; C12N 15/1136; A61P 3/00; A61P 9/00; A61K 39/3955

(57) ABSTRACT

The present invention relates to methods and pharmaceutical compositions for the treatment of age-related cardiometabolic diseases. The inventors identified osteopontin (OPN) as a critical mediator of adipose tissue remodeling and senescence in obesity and extends this observation to related co-morbidities such as cardiomyopathy. Said result raises the possibility that inhibition of OPN activity may be of value in the prevention of cardiometabolic disease, in particular metabolic cardiomyopathy for which no specific treatment is yet available. In particular, the present invention relates to a method of treating an age-related cardiometabolic disease in an elderly subject in need thereof comprising administering to the subject a therapeutically effective amount of an osteopontin (OPN) inhibitor.

### FIELD OF THE INVENTION

The present invention relates to methods and pharmaceutical compositions for the treatment of age-related cardiometabolic diseases.

### BACKGROUND OF THE INVENTION

Obesity affects ˜23% of women and ˜20% of men in WHO European

(http://www.euro.who.int/en/health-topics/noncommunicable-diseases/obesity). Obesity is associated with many co-morbidities and leads to premature death from diabetes and cardiovascular disorders. Obesity is also coupled to an acceleration of multiple age-related diseases. It is projected that obesity and diabetes will be the major contributors to cardiovascular mortality and morbidity in the 21^{st} century and will compromise healthy aging. Osteopontin (OPN), a secreted extracellular matrix protein, is involved in cell migration and adhesion, macrophage activation, inflammation and matrix remodeling (J. Clin. Invest. 117:2877-88; 2007) and is over-expressed in obesity and aging. Previous data show that antibody mediated neutralization of OPN significantly reduces insulin resistance in obesity, decreases obesity-associated inflammation in adipose tissue and reverses signal transduction related to insulin resistance and glucose homeostasis (Diabetes 59:935-946, 2010). Recent data also suggest the role of OPN in cardiac dysfunction under diabetic conditions and indicates that lack of OPN protein can be beneficial in preserving function of the diabetic heart (Am J Physiol Heart Circ 292: H673-83, 2007). Pharmacological OPN inhibition has been taught for the treatment of diabetes (WO2008131094).

### SUMMARY OF THE INVENTION

The present invention relates to methods and pharmaceutical compositions for the treatment of age-related cardiometabolic diseases. In particular, the present invention is defined by the claims.

### DETAILED DESCRIPTION OF THE INVENTION

The inventors propose a new hypothesis that obesity “triggers” adipose tissue (AT) senescence which in turn is crucial to the pathogenesis of obesity co-morbidities by affecting neighbouring and distant tissues. They identified osteopontin (OPN) as a critical mediator of AT remodeling and senescence in obesity and extends this observation to related comorbidities such as cardiomyopathy. Said result raises the possibility that inhibition of OPN activity may be of value in the prevention of cardiometabolic disease, in particular metabolic cardiomyopathy for which no specific treatment is yet available.

Accordingly a first object of the present invention relates to a method of treating an age-related cardiometabolic disease in an elderly subject in need thereof comprising administering to the subject a therapeutically effective amount of an osteopontin (OPN) inhibitor.

As used herein, the term “elderly subject” refers to an adult patient sixty-five years of age or older.

In some embodiments, the elderly subject is obese. The term “obesity” refers to a condition characterized by an excess of body fat. The operational definition of obesity is based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m2 to less than 25 kg/m².

As used herein, the term “cardiometabolic disease” has its general meaning in the art and relates to cardiovascular diseases associated with metabolic syndrome, such as obesity, diabetes/insulin resistance, hypertension and dyslipidemia. The term “cardiometabolic diseases” refers to cardiac consequences of metabolic syndrome such as atherosclerosis, coronary heart disease, obesity-associated heart disease, insulin resistance-associated heart disease, hypertensive heart disease, cardiac remodeling, heart failure and cardiometabolic eases disclosed in Hertle et al, 2014; Hua and Nair, 2014; U.S. Pat. Application No. 2012/0214771 and International Patent Application No. 2008/094939. As used herein, the term “age related cardiometabolic disease” relates to any cardiometabolic disease which has a factor of its etiology the age of the subject. It will be understood that age may only be one of a number of factors, which combined, result in the development of the disorder.

In some embodiments, the method of the present invention is particularly suitable for the treatment of metabolic cardiomyopathy. In particular, the osteopontin inhibitor is particularly suitable for rescuing age-related cardiac remodeling, reducing cardiac fibrosis and improving cardiac function.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “osteopontin” or “OPN” has its general meaning in the art. The term “osteopontin” is used interchangeably with “OPN,” “SPP1,” “Eta-1,” sialoprotein 1 or 44K BPP (bone phosphoprotein). Accordingly an “osteopontin inhibitor” refers to any compound that is capable of inhibiting the activity or expression of osteopontin. As used herein, the term “osteopontin activity” includes any biological activity mediated by osteopontin such as described in the EXAMPLE. In particular, the osteopontin inhibitor of the present invention is particular suitable for abrogating adipose tissue senescence; reducing adipose tissue macrophage accumulation, attenuating adipose tissue inflammation, and protecting myocardial function. Examples of osteopontin inhibitors include but are not limited to polypeptides such as dominant-negative protein mutants, peptidomimetics, antibodies, ribozymes, antisense oligonucleotides, or other small molecules which specifically inhibit the activity or expression of osteopontin.

In some embodiments, the osteopontin inhibitor of the present invention is a small organic molecule such as Agelastatin A (AA). Agelastatin A is an oroidin alkaloid extracted from an axinellid sponge, Agelas dendromorpha. The bioactive agelastatins are members of pyrrole-imidazole family of marine alkaloids that possess a tetracyclic molecular framework incorporating C4-C8 and C7-N12 bond connectivities potentiating numbers of differently functional derivatives (Chem Sci. 2010, 1, 561). AA can be produced by any well known method in the art and in particular by the method disclosed in Yoshimitsu T. et al, Org Lett. 2008, 10, 5457 & Org Lett. 2009, 11, 3402. Agelastatin A has the formula of:

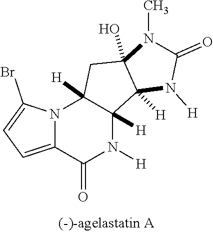

In some embodiments, the osteopontin inhibitor is an antibody. As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. In some embodiments, the antibody of the present invention is a single chain antibody. As used herein the term “single domain antibody” has its general meaning in the art and refers to the single heavy chain variable domain of antibodies of the tune that can be found in Camelid mammals which are naturally devoid of light chains. Such single domain antibody are also “nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

In some embodiments, the osteopontin inhibitor is an inhibitor of osteopontin expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of OPN mRNA by binding thereto and thus preventing protein translation or increasing mRNA degradation, thus decreasing the level of OPN, and thus activity, in a cell. For example, antisense oligonucleotides of at least about 15 bases and complementary to unique regions of the mRNA transcript sequence encoding OPN can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. OPN gene expression can be reduced by contacting a subject or cell with a small double stranded RNA (dsRNA), or a vector or construct causing the production of a small double stranded RNA, such that OPN gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing OPN. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

By a “therapeutically effective amount” of the osteopontin inhibitor as above described is meant a sufficient amount to provide a therapeutic effect. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the osteopontin inhibitor is administered to the subject in the form of a pharmaceutical composition. Typically, the osteopontin inhibitor may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The osteopontin inhibitor can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: OPN expression is by aging mainly in up-regulated in adipose tissues but also in the heart. Relative OPN gene induction by aging in tissues. Fold induction levels are compared to those in young WT organs. Data are mean±SEM, n=3-9, unpaired-t test: ***p<0.001, **p<0.01, and *p<0.05.

FIG. 2: Aging and metabolic stress promoted OPN (Spp1) promoter demethylation. Epigenetic analysis (CpG methylation level) also showed significant OPN promoter lesion activation by aging (7 months old) and high-fat diet (HFD) in heart tissue. as compared with young (3 months old) fed with chow diet mice. Unpaired-t test: p=5.20E−06

FIG. 3: Body-weight (BW) comparison of wild-type (WT) and OPN−/− mice at two ages (young: 3 months old and aged: 12 months old). No difference between young WT and OPN−/− mice was observed. With age, BW significantly increased in both groups but at a higher level in OPN−/− mice as compared to WT mice. n=10-14 each group, one-way ANOVA: ***p<0.001, ns indicates no significance by Bonferroni's post-hoc analysis.

FIG. 4: Glucose tolerance test (GTT) by intraperitoneal administration in aged (12 months old) WT mice. Aged OPN−/− mice showed significantly lower glucose values as compared to aged WT mice. n=6 each group, two-way ANOVA: p=0.0073 compared to WT mice, ***p<0.001 by Bonferroni's post-hoc analysis.

FIG. 5: Insulin tolerance test (ITT) by intraperitoneal administration in aged (12 months old) WT mice. WT and OPN−/− mice were comparable despite a trend of higher insulin sensitivity in OPN−/− mice. n=6 each group, two-way ANOVA: p=0.0833 compared to WT mice.

FIG. 6: Heart weight/tibia length ratio (HW: mg/TL: mm) of WT and OPN−/− mice at two ages (young: 3 months old and aged: 12 months old). No significant difference in HW/TL was observed in young WT and OPN−/− mice. With age, HW/TL significantly increased in WT mice but not in OPN−/− mice. n=5-13 each group, one-way ANOVA: ***p<0.001, **p<0.01, ns indicates no significance by Bonferroni's post-hoc analysis.

FIG. 7: Myocardial fibrosis ratio (%) of WT and OPN−/− mice at two ages (young: 3 months old and aged: 12 months old). No significant difference in interstitial fibrosis was observed in young WT and OPN−/− mice. With age, interstitial fibrosis significantly increased in aged WT mice but not in OPN−/− mice. Heart samples were paraffin-embedded and stained with Sirius-Red. Interstitial fibrosis area was analyzed with Image J software. Twelve to 16 magnified pictures were analyzed in each group. One-way ANOVA: ***p<0.001, ns indicates no significance by Bonferroni's post-hoc analysis.

FIG. 8: Phosphorylated Smad3 (pSmad3) myocardial positive ratio. Sections of heart were immunohistologically stained for pSmad3 and counterstained with DAPI. Positive ratio was calculated as pSmad3 positive cells/total DAPI positive nuclei (%). Data are mean±SEM. Analysis were done with 9 pictures from 3 mice each group. One-way ANOVA: *p<0.05, **p<0.01 by Bonferroni's post-hoc analysis.

FIG. 9: Cardiomyocyte cross-sectional area (CSA: □m²) of WT and OPN−/− mice at two ages (young: 3 months old and aged: 12 months old). No difference in CSA was observed in young WT and OPN−/− mice. With age, CSA significantly increased in both WT and OPN−/− mice, but was significantly blunted in OPN−/− mice. Heart samples were paraffin-embedded and stained with wheat germ agglutinin conjugated with fluorochrome. Cross-sectional cell surface area was analyzed with Image J software. At least 200 cells were analyzed from each group. One-way ANOVA: ***p<0.001, ns indicates no significance by Bonferroni's post-hoc analysis.

FIG. 10: Myocardial strain rate (anterior wall) was evaluated in WT and OPN−/− mice at two ages (young: 3 months old and aged: 12 months old). Aged OPN−/− mice showed preserved strain rate compared to aged WT mice. n=5-10 each group, One-way ANOVA: *p<0.05, **p<0.01,ns indicates no significance by Bonferroni's post-hoc analysis.

FIG. 11: Association plot between SR values (systolic function) and importance of interstitial fibrotic deposition. Note that there is a strong negative correlation between fibrosis and cardiac systolic function.

FIG. 12: Myocardial induction of TGF□-1 (fibrosis-related gene) expression level by aging. Compared to WT mice, OPN deficiency attenuated myocardial fibrotic gene induction by aging. Data are mean±SEM, n=6. Unpaired t-test: ***p<0.001.

FIG. 13: Agelastatin A (AA) treatment diminished senescent cells in the aged visceral adipose tissue. Representative immunostaining of p16 positive cells (white) in visceral adipose tissue from aged WT mice treated with vehicle (upper panel) or AA (lower panel). Right-sided bar graph shows quantification (%) of p16 positive cells/total nuclei. Unpaired t-test: ***p<0.001.

FIG. 14: Glucose tolerance test (GTT) under Agelastatin A (AA) treatment in aged (12 months old) WT mice. GTT by intraperitoneal glucose administration was performed at baseline and 4 weeks with AA treatment. There was no difference between vehicle and AA group at baseline. AA group showed significantly lower glucose values after 4 weeks of treatment. n=6 each group, two-way ANOVA: Bonferroni's post-hoc analysis results as indicated in the figure.

FIG. 15: Insulin tolerance test (ITT) under AA treatment in aged (12 months old) WT mice. ITT by intraperitoneal insulin administration was performed at baseline and 4 weeks with AA treatment. There was no difference between vehicle and AA group at baseline. AA group showed significantly lower glucose values after 4 weeks of treatment. n=6 each group, two-way ANOVA: Bonferroni's post-hoc analysis results as indicated in the figure.

FIG. 16: Myocardial fibrosis ratio (%) after 4 weeks with AA treatment in aged (12 months old) WT mice. The increase in interstitial fibrosis observed in aged mice treated with vehicle was rescued by AA treatment in aged WT mice. Heart samples were paraffin-embedded and stained with Sirius-Red. Interstitial fibrosis area was analyzed with Image J software. Ten to 14 magnified pictures were analyzed in each group. One-way ANOVA: ***p<0.001 by Bonferroni's post-hoc analysis.

FIG. 17: Myocardial strain rate (anterior wall) was measured every week during vehicle or AA administration in aged (12 months old) WT mice. AA treated mice showed significant and sustained improvement of strain rate as compared with vehicle treated mice. n=8 each group, two-way ANOVA: p=0.0002 compared to vehicle group, ***p<0.001 by Bonferroni's post-hoc analysis.

FIG. 18: In vivo hemodynamics analysis after AA treatment in aged (12 months old) WT mice. In vivo cardiac function was evaluated by intra-cardiac pressure analysis. Both systolic function (dP/dtₘₐₓ: left panel) and diastolic function (dP/dtₘᵢₙ: right panel) were significantly improved by 4 weeks of AA treatment, which were comparable to that of young mice. There was no difference in heart rate in the both group (data not shown). n=7-8 each group, one-way ANOVA: **p<0.01, ***p<0.001 by Bonferroni's post-hoc analysis.

FIG. 19: Agelastatin A treatment attenuated myocardial TGF□-1 (fibrosis-related gene) expression level by aging. Data are mean±SEM, n=6. Unpaired t-test: **p<0.01.

### EXAMPLE

Material & Methods

Mice:

C57/BL6JRj mice (referred as wild-type: WT) were purchased from Janvier Labs,

France. Osteopontine knockout mice (B6.129S6(Cg)-Spp1^{tm1Bth}/J) were purchased from Jackson laboratory (ME, USA), which had been back-crossed with C57/BL6J mice more than 10 generations, and kept as homozygous (referred as OPN−/−). All mice were housed in a the specific pathogen free platform at a constant temperature (22° C.), with a 12-hour light-dark cycle and unrestricted access to a chow diet (CD, A0310, Safe Diets, France) and water. Chow diet composition corresponded to 13.5% of fat, 61.3% of carbohydrate and 25.2% of protein. During follow-up, animals underwent a monthly body-weight (BW), metabolic and echocardiography evaluations. Before sacrifice, animals underwent a hemodynamic evaluation. All animal experiments were approved by the Institutional Animal Care and Use Committee of the French National Institute of Health and Medical Research (INSERM)-Unit 955, Créteil, France (ComEth 15-001).

Metabolic Tolerance Tests:

Glucose Tolerance Test (GTT): Mice were fasted for 6-7 hours before the study, and then given glucose solution intraperitoneally (1.5 mg/g body weight). Blood samples were collected from tail vein at baseline, 15, 30, 60, 90, 120 minutes after injection, and glucose concentration was measured with an automatic glucometer (Accu-Chek Performa, Roche, Germany).

Insulin Tolerance Test (ITT): Mice were fasted for 6-7 hours before study, and then given insulin solution intraperitoneally (0.5 mU/g of body weight, Umelin, Lilly, France). Blood samples were collected from tail vein at baseline, 30, 60, 90, 120 minutes after injection, and glucose concentration was measured as above.

Transthoracic Echocardiography Analysis

Mice were regularly manipulated in order to avoid any stress related to echocardiography assessment. Transthoracic echocardiography (TTE) was performed in non-sedated mice in order to avoid any depression effect of anesthetic agents. Mice were carefully caught by the left hand and placed in supine position. Data acquisition and analysis were performed monthly. Images were acquired from a parasternal position at the level of papillary muscles using a 13-MHz linear-array transducer with a digital ultrasound system (Vivid 7, GE Medical System). Left ventricular diameters and ejection fraction, anterior and posterior wall thicknesses were serially obtained from gray-scale M-mode acquisition. Peak systolic values of strain rate (SR) in the anterior and posterior wall were obtained using Tissue Doppler Imaging (TDI). TDI loops were acquired from the same parasternal view at a mean frame rate of 450 i/s and a depth of 1 cm. The Nyquist velocity limit was set around 12 cm/s. Strain rate analysis was performed offline by an observer blinded to the groups using the EchoPac Software (GE Medical). The region of interest size was set at 0.2 mm and temporal smoothing filters were turned off for all measurements. Because slight respiratory variations exist, we averaged peak systolic SR on 8 consecutive cardiac cycles. Reproducibility of echocardiography measurements has already been published.

Hemodynamic Analysis and Blood Sample Collection

Analyses were performed just before sacrifice and harvesting the organs. Hemodynamic measurements were performed in mice placed on a homeothermic operating table under 1.5% isoflurane anesthesia in spontaneous breathing. A 1.4-Fr microcatheter (Millar Instruments, Houston, USA) calibrated manually before each experiment, was inserted via the right carotid artery into the aorta for measurement of systolic and diastolic pressure, and then advanced into the LV for measurement of end-diastolic and end-systolic pressures, peak rates of isovolumic pressure development (max. dP/dt) and pressure decay (min. dP/dt). Data were collected after at least 10 min of steady state, using the lowest isoflurane concentration tolerated. Data were analyzed using the IOX software (EMKA, France). After in vivo hemodynamic analysis, blood was taken from carotid artery into EDTA-2Na coated tubes ( ) centrifuged at g for 10 minutes, and kept in −80° C. deep freezer. The mice were euthanized by neck dislodgement immediately.

Tissue Processing and Histology

Dissected organs were fixed with 4% formaldehyde solution (Sigma-Aldrich) immediately and subjected to paraffin-embedding after at least 1 week fixation. The sections were deparaffinized using xylene and a graded series of ethanol dilutions. Adipose tissue sections (5 μm thickness) were stained with hematoxyline & eosin (Sigma-Aldrich, France) for crown-like structure (CLS) number and adipocyte size measurement.

Heart tissue sections were stained either with wheat germ agglutinin (WGA; plasma membrane staining, Alexa Fluor 488 conjugated) for cardiomyocyte size (cross-sectional area) or Sirius Red for interstitial fibrosis measurement, respectively.

For immuno fluorescent staining, deparaffinized and hydrated sections were then subjected to heat-mediated antigen retrieval (Citrate buffer pH6.0, Dako) and blocking with 30% goat serum. Primary antibodies were applied with antibody diluent (Dako) for over-night at 4° C. in humidified chamber. Corresponding 2^{nd} antibodies conjugated with fluorescent dyes were used to visualize with DAPI nucleus counter-staining.

The pictures with hematoxyline & eosin and Sirius Red staining were captured using a Zeiss Axioplan 2 Imaging microscope. Fluorescent images were taken using a Zeiss Axioplan M2 Imaging microscope. For adipocyte size and cardiomyocyte size, at least 200-300 cells per sample were traced and quantified surface area. For interstitial fibrosis, 3-5 images per sample were measured red area normalized by total surface area. All data were measured with the ImageJ software (NIH).

SA-β-Gal Staining

Dissected organs were directly subjected to staining solution (1 mg/ml Xgal, Sigma-Aldrich) as a whole-mount (heart) or in 5 mm pieces (fat pads), and kept at 37° C. for 12 hours. The images were taken by GR digital camera (RICOH, Japan). After taking pictures, stained samples were kept in 4% formaldehyde solutions (Sigma-Aldrich) and subjected to paraffin-embedding for further analysis.

Quantitative PCR

Total RNA was extracted from 20 mg (heart) or 80 mg (adipose tissue) powdered samples with RNA easy fibrous kit (Qiagen). RNA from cultured macrophages was extracted with RNA easy mini kit (Qiagen) as manufacturer's instruction. First strand DNA synthesis was synthesized from 0.5-1.0 μg total RNA with High-Capacity cDNA Reverse Transcription Kit and a thermal cycler SimpliAmp (Applied Biosystems) according to manufacturer's instruction. Quantitative PCR was performed and analyzed with TaqMan® Universal PCR Master Mix and StepOne™ Real-Time PCR System (Applied Biosystems). B-actin was used as internal control.

Western Blot Analysis

Twenty mg of powdered myocardium or 80 mg of adipose tissues was homogenized in “Platonic” urea lysis buffer containing 7 M urea, 10% glycerol (v/v), 10 mM Tris-HCl (pH6.8), 1% SDS, 1 mM dithiothreitol, supplemented with protease and phosphatase inhibitor cocktail tablets (Pierce). After vortexing, lysates were sonicated, passed through a 21G needle at least 6 times, rotated for 30 minutes at 4° C. and spun to obtain supernatant. Protein concentration was adjusted according to Bradford method (Bio-rad). Denatured total protein (20 μg) was loaded to 10% SDS-PAGE. Separated proteins were transferred to PVDF membranes (Invitrogen), blocked in 5% milk in TBS-Tween (0.1%) and probed for selected proteins with a specific primary antibody at 4° C. overnight. Primary antibodies were used against osteopontin (ab8448, Abcam), p16 (250804, Abbiotec), p21 (HUGO291, EuroMabNet), p53 (ab31333, Abcam), and β-actin (ab16039, Abcam), followed by incubation with the corresponding HRP-conjugated secondary antibody (Abcam). Densitometric quantification was normalized to β-actin levels in each sample.

Malondialdehyde Measurements

Myocardial and fat pads lipid peroxidation was assessed by measuring malondialdehyde levels. Briefly, hearts were pulverized with mortar and pestle using liquid nitrogen and 25-30 mg of the powder was mixed with 2 volumes of ice cold 10% (w/v) TCA. Samples were then sonicated two times for 30 seconds followed by passing them through 21 G needle at least 6 times before centrifugation at 13000 rpm for 5 min at +4° C. An aliquot of the supernatant was reacted with an equal volume of 0.67% (w/v) TBA in a boiling water bath for 10 minutes. Samples were allowed to cool down before absorbance was read @ 532 nm. The concentration of MDA was calculated based on the c value of 153000 and normalized to wet weight of the sample.

Adipose Tissue Macrophage Flow Cytometric Analysis (FACS)

Dissected adipose tissues were kept on ice and minced to 1-3 mm pieces in heparinized cold PBS. After snap vortexing, PBS was removed and tissues were digested with collagenous solution (Sigma-Aldrich) at 37° C. water bath with gently shaking for 20 min. Digested tissue was then passed through 70 μm mesh and centrifuged to spin down stromal vascular fraction (SVF). After RBC lysis, the SVF cells were suspended in PBS supplemented 5% FCS, and stained with appropriate antibodies for surface marker and isotype controls for 30 min at 4° C. in the dark chamber: F4/80 (BM8, Biolegend), CD1 lb (M1/70, Biolegend), CD11c (N418, Biolegend), CD206 (MR5D3, AbDSerotec). Living and dead cell discrimination was performed with 7-AAD staining (Molecular Probes). For intracellular staining, SFV cells stained with surface markers were then fixed and permeabilized with Cytofix/Cytoperm solution (BD Bioscience). The cells were then stained with Alexa 488 conjugated p16 antibody (2D9Al2, Abcam), FITC-conjugated Ki67 antibody (SolA15, eBioscience) or corresponding isotype controls. Flow cytometry was performed with LSRII and cell sorting was done with INFLUX (BD Bioscience). Data was evaluated with FlowJo (TreeStar).

Generation of Bone-Marrow Derived Macrophages

Mice bone marrow was isolated from femurs and tibias from WT and OPN−/− mice. After RBC lysis and passed through 70 μm mesh, bone marrow cells were plated with RPMI 1640 medium (Gibco) supplemented with 10%FCS and 30 ng/ml MCSF (R&D systems). Five days after differentiation into macrophages (bone marrow derived macrophage: BMDM, M0), the cells were dissolved, counted and re-plated at 1.5×10⁵/well in 12-well non-coated plate. The cells were given 10 ng/ml LPS (Sigma-Aldrich) and 50 ng/ml IFNg, or 20 ng/ml IL4 (Peprotech, NJ, USA) to further differentiate into M1 or M2 macrophages respectively.

Bone Marrow Derived Macrophage (BMDM) Stimulation by Osteopontin

Modulation of BMDM by OPN protein was evaluated as previously described with minor modification (Circ Res 2010; 107:1313-1325). Briefly, 12 well plates were coated with 0.1% gelatin (Sigma-Aldrich) or 3.5 μg/mL of mouse recombinant OPN (R&D systems) for 20 h at 4° C. and then stabilized with 0.5% polyvinylpyrrolidone (Sigma-Aldrich) for 1 h at room temperature. BMDM (1.5×10⁵/well) were re-plated in RPMI 1640 medium supplemented with 10% FCS and 30 ng/ml MCSF. Twenty-four hours after passage, the cells were harvested for RNA extraction.

In Vivo Macrophage Depletion

The total of 17 male wild type C57BL/6J mice, 14 months of age and weighed 30˜36 g, were used. Before in vivo macrophage depletion, mice were undergone baseline assessment of metabolic and cardiac functions (GTT, ITT and echocardiography). The mice were then injected intraperitoneally with 70 mg/kg clodronate in liposomes or PBS control liposomes (ClodLip BV, Amsterdam, the Netherlands) every 4 days for 4 weeks (70% dose used in reference: Diabetes 2014; 63:1698-1711). Their body weights were recorded at the same time. After final metabolic and cardiac assessments, the mice were subjected to in vivo hemodynamic analysis and euthanized by neck-dislodgement. The organs and bloods were harvested and processed for further evaluations as written elsewhere.

Osteopontin Inhibition by Small Molecule

The total of 20 male wild type C57BL/6J mice, approximately 11 months of age and weighed 28˜35 g, were used. AgelastatinA (AA), small organic molecule known to inhibit OPN production in vivo, was synthesized and kindly provided by Professor Takehiko Yoshimitsu at Graduate School of Pharmaceutical Sciences, Osaka University, Osaka, Japan. After baseline assessment of metabolic and cardiac functions (GTT, ITT and echocardiography), the mice were given 1.5 mg/kg of AA or vehicle only (2-Hydroxypropyl-β-cyclodextrinonly, Sigma-Aldrich, MI, USA) intraperitoneally every 4 days for 4 weeks. Their body weights were recorded at the same time. This protocol was developed with informative advices from Pro.Yoshimitsu. After final metabolic and cardiac assessments, the mice were subjected to in vivo hemodynamic analysis and euthanized by neck-dislodgement. The organs and bloods were harvested and processed for further evaluations as written elsewhere.

Epididymal Adipose Tissue Resection

The total of 20 male wild type C57BL/6J mice, approximately 11 months of age and weighed 28˜36 g, were used. Before epididymal fat resection, mice were undergone baseline assessment of metabolic and cardiac functions (GTT, ITT and echocardiography).The surgical procedures were performed as described in a previous study (Brain, Behavior, and Immunity 2015; 50: 221-231). Briefly, the mice were anesthetized with isoflurane and a 1 cm single abdominal midline incision was made. Bilateral epididymalfat pads were lifted from the peritoneal cavity onto a sterilized and humidified surgical drape, dissected with an electronic scalpel, and removed without damaging the testicular blood supply. The sham operation was performed in the same manner without fat pads removal. The abdominal peritoneum was closed with prolynsutures and the skin was closed with silk sutures (Angiotech, PA, USA).The mice were let to recover from surgery and regain body weight. Their body weights were recorded weekly. All the mice remained alive without significant body weight-loss. Five weeks after surgery, final metabolic and cardiac assessments were performed. Then the mice were subjected to in vivo hemodynamic analysis and euthanized by neck-dislodgement. The organs and bloods were harvested and processed for further evaluations as written elsewhere.

Statistical Analysis

All data are presented as mean+SEM. Difference between two groups was analyzed by unpaired t-test. Comparisons between multiple groups were done using one- or two-way ANOVA followed by a post hoc Bonferroni test. P-value<0.05 was considered significant.

Results

We established high fat diet (HFD) murine models with serial metabolic and myocardial follow-up studies focusing on both adipose tissue (AT) remodeling and cardiac function to prove the concept that obesity induces AT senescence and to demonstrate that increased OPN expression and cell senescence are associated in HFD models. In particular, C57BL6 mice (wild-type: WT) were fed with control diet (CD) or HFD (60% fat, 7% sucrose) for 20 weeks. As previously reported, HFD mice developed impaired glucose tolerance. Time dependent cardiac dysfunction was evident by serial cardiac echography and in vivo hemodynamic measurement in HFD group. Interestingly, HFD mice developed increased myocardial senescent marker expression.

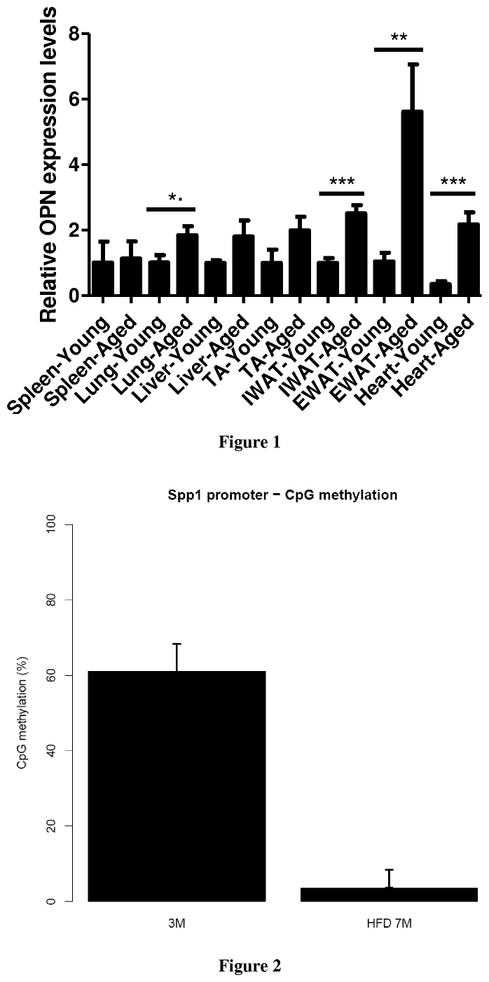

On the other hand, we demonstrated for the first time aging-induced increase of myocardial OPN expression, suggesting strong association between OPN upregulation and tissue senescence in myocardial age-related dysfunction in addition to obesity-induced cardiac dysfunction. Our original data show that OPN expression is up-regulated specifically in the heart and adipose tissues by aging (FIG. 1), and epigenetic analysis further confirmed significantly increased promoter activity in heart according to aging and metabolic stress (FIG. 2).

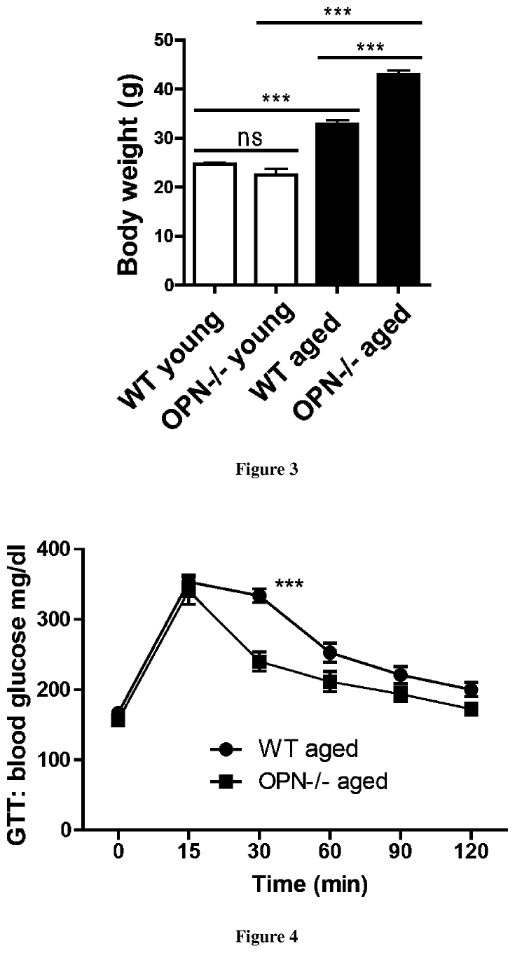

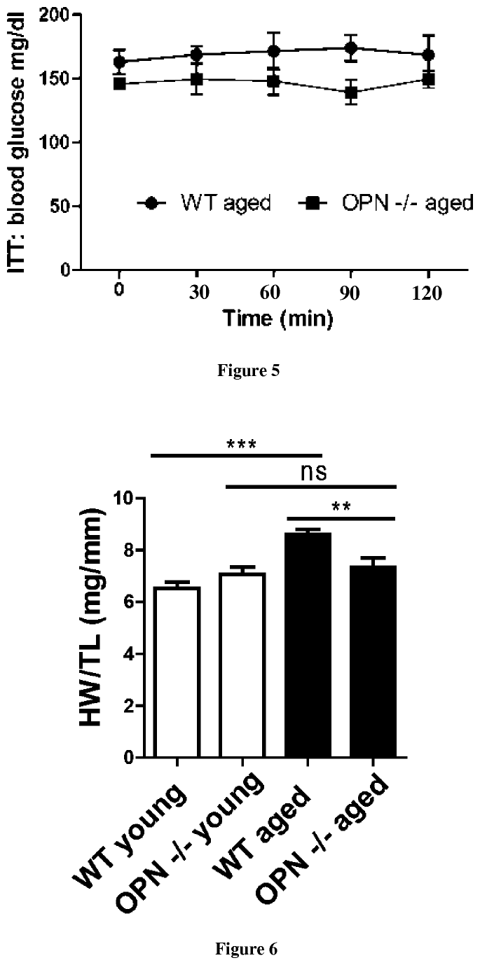

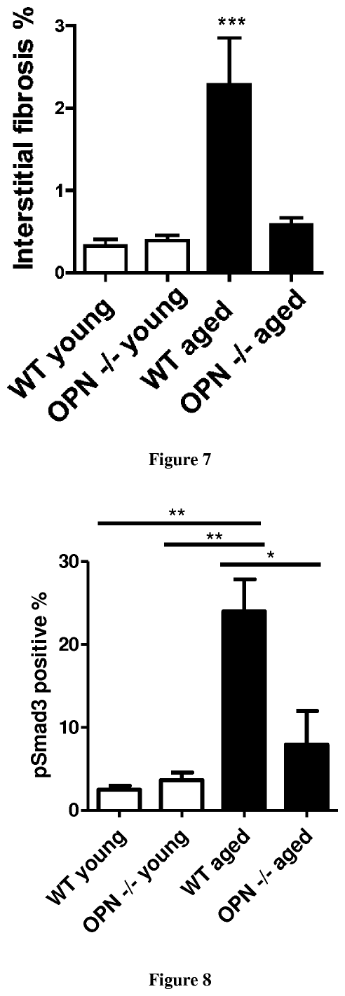

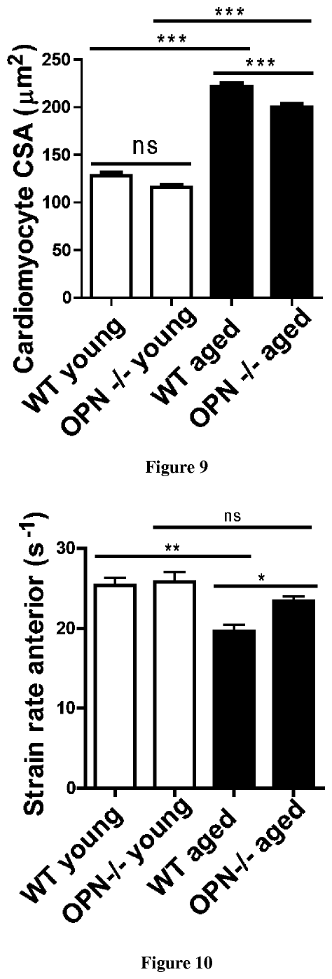

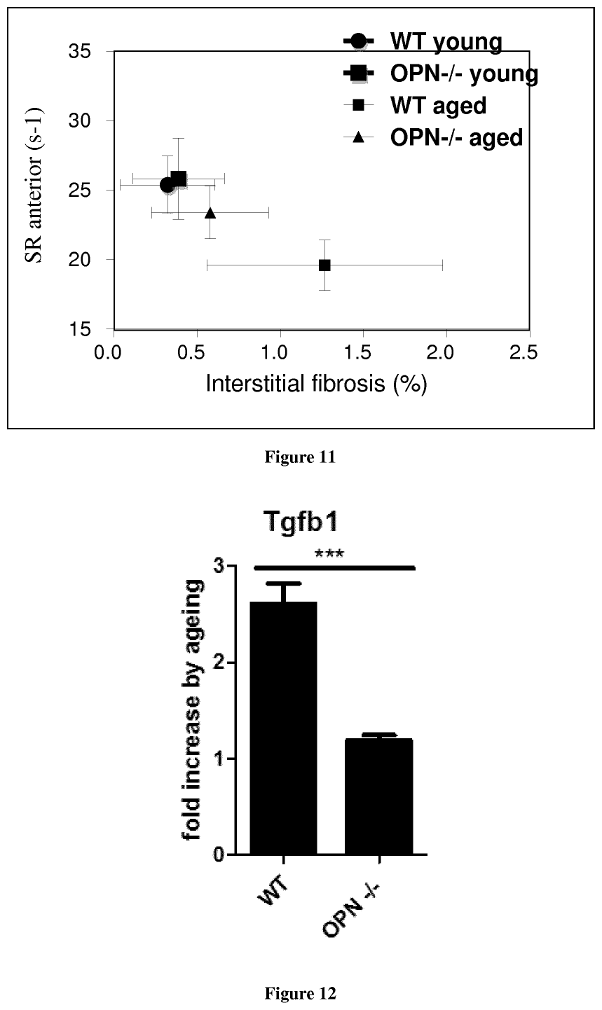

We further have demonstrated a cardiometabolic improvement in aged OPN KO mice. To explore a role for OPN in age-related cardiometabolic abnormalities OPN levels were measured in metabolic tissues (liver, tibialis anterior (TA) muscle, inguinal (IWAT) and epididymal (EWAT) white AT and heart) in young (2-3 months old) and aged (12-13 months old) male WT mice. Although there was a trend of upregulation in all tissues, OPN was mainly increased in IWAT and EWAT. Moreover OPN expression (immunostaining) was also increased both in adipose tissue and myocardium. To examine whether blocked OPN induction could protect against age-related cardiometabolic dysfunction, aged OPN KO mice and WT littermate controls were compared to young ones: body weight gain was greater in OPN KOs than WT mice (FIG. 3). All fatty pads (perirenal: PRWAT, IWAT, EWAT) were heavier in aged OPN KO mice than in age-matched WTs, with larger adipocyte size. Interestingly, this was associated with improved glucose metabolism in aged OPN KO mice compared to WTs (glucose and insulin tolerance tests: GTT & ITT, FIGS. 4 and 5). Further studies of EWAT revealed increased senescence (β-Gal staining and p16+ adipose tissue macrophages: ATMs, and number of crown-like structures (adipocyte death with surrounding inflammation, in aged WT compared to OPN KO. Importantly, OPN protein expression was greatly induced in ATMs extending to the inter-adipocyte extracellular space FACS analysis identified significantly increased p16 expression in ATMs by aging. Age-related cardiac remodeling (increased heart weight to tibia length: HW/TL, fibrosis, Smad3 phosphorylation and cardiomyocyte hypertrophy) in WT mice was partially rescued by OPN deficiency (FIGS. 6, 7, 8 and 9). This was associated with improved cardiac function (strain rate by echocardiography) in aged OPN KO hearts compared to WTs (FIG. 10). Interestingly, interstitial fibrosis showed clear negative correlation with systolic function (FIG. 11). Myocardial TGFb1 expression induction was also attenuated in the OPN KO mice (FIG. 12).

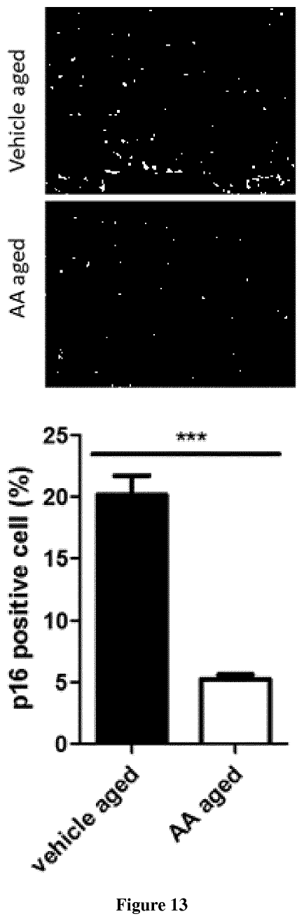

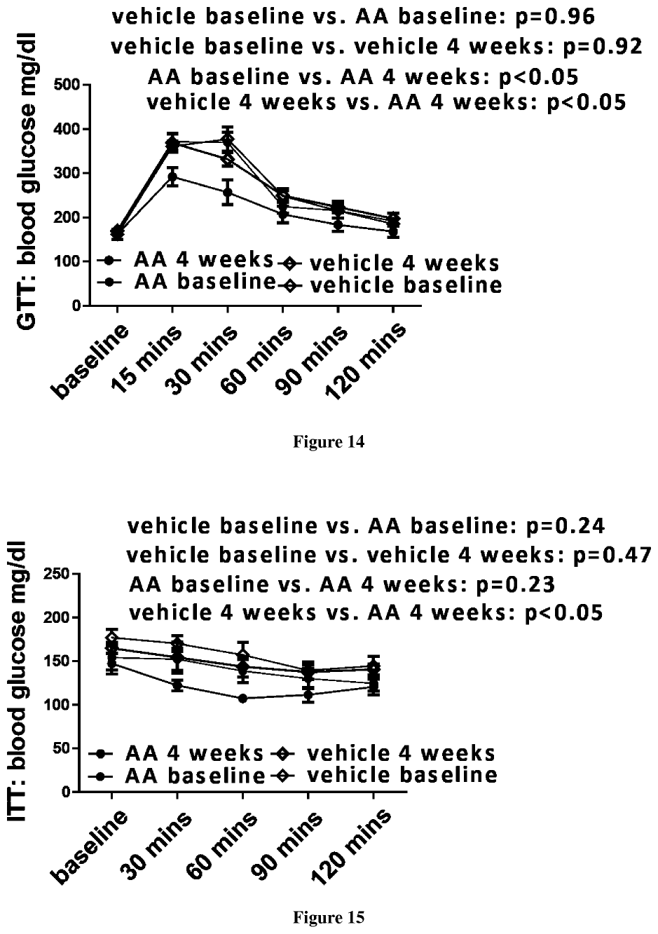

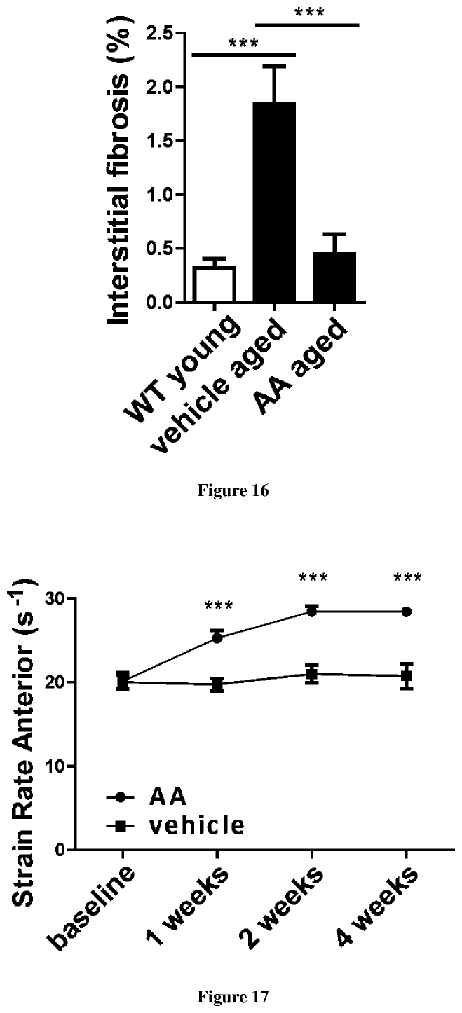

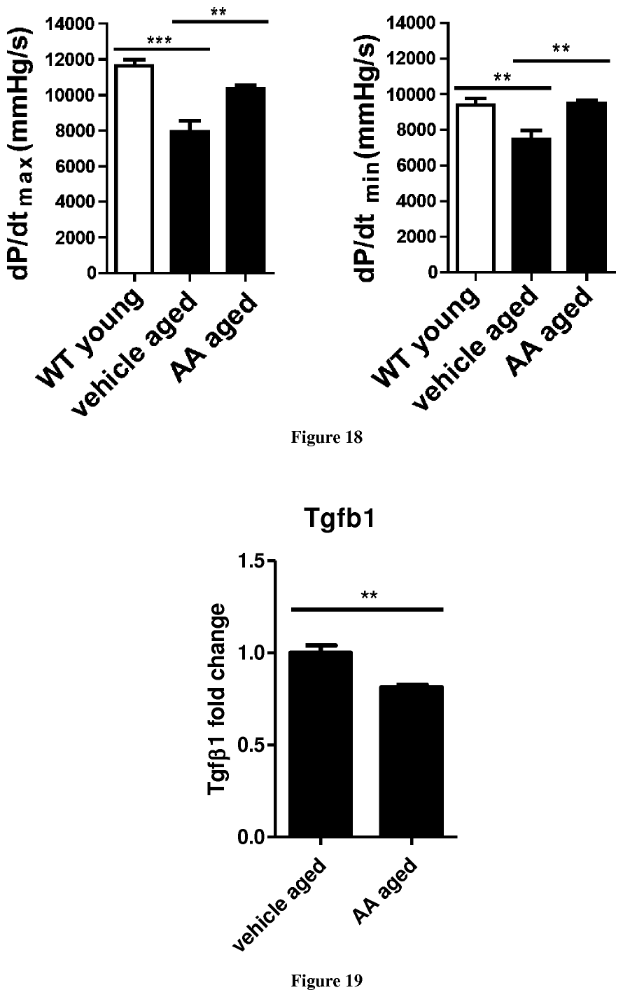

Encouraged by these findings, we wanted to assess whether pharmacological inhibition of OPN with Agelastatin A (AA) could recap the cardiometabolic improvement in aged mice observed above and is protective against obesity & age comorbidities. We have administered an OPN inhibitor Agelastatin A at a dose of 2.5 mg/Kg in 4 day per 2 weeks for a period of 2 months in old (12 months old) and have assessed the effects on glucose and insulin sensitivity, AT remodeling and senescence, myocardial function, cardiac hemodynamics (dP/dtmax/min) and remodeling (interstitial fibrosis and cardiomyocytes size).When one year-old male WT mice were treated with AA they showed significantly less senescent cells in the visceral adipose tissue (FIG. 13), and improved metabolism (GTT & ITT, FIGS. 14 and 15), cardiac structure (fibrosis, FIG. 16) and function (strain rate, FIG. 17 & in vivo hemodynamic analysis, FIG. 18) over vehicle. In parallel, Tgfb1 expression was also attenuated by AA treatment (FIG. 19).

In conclusion we demonstrated that OPN inhibition is particularly suitable for the prevention of age-related cardiometabolic dysfunction in particular metabolic cardiomyopathy.

### REFERENCES

Throughout this application, various references describe the state of the art to which this invention pertains. The disclosures of these references are hereby incorporated by reference into the present disclosure.

1. A method of treating an age-related cardiometabolic disease in an elderly subject in need thereof comprising administering to the subject a therapeutically effective amount of an osteopontin (OPN) inhibitor.

2. The method of claim 1 wherein the elderly subject is obese.

3. The method of claim 1 wherein the age-related cardiometabolic disease is a metabolic cardiomyopathy.

4. The method of claim 1 wherein the osteopontin inhibitor is a small organic molecule.

5. The method of claim 1 wherein the osteopontin inhibitor is an antibody having specificity for osteopontin.

6. The method of claim 1 wherein the osteopontin inhibitor is an inhibitor of osteopontin expression.

7. The method of claim 4, wherein the small organic molecule is Agelastatin A (AA).

8. The method of claim 6, wherein the inhibitor of osteopontin expression is a siRNA or an antisense oligonucleotide.
